(19) United States
(12) Patent Application Publication
(10) Pub. No.: US 2022/0273223 A1
(43) Pub. Date: Sep. 1, 2022

(54) DEVICE FOR DETERMINING A PIECE OF INFORMATION RELATING TO A CARDIAC DECOMPENSATION STATE

(71) Applicants: UNIVERSITE GRENOBLE ALPES, Saint-Martin-d'Hères (FR); CENTRE NATIONAL DE LA RECHERCHE SCIENTIFIQUE, PARIS (FR); CENTRE HOSPITALIER UNIVERSITAIRE GRENOBLE ALPES, GRENOBLE (FR); GRENOBLE INP, GRENOBLE (FR); SENTINHEALTH, PARIS (FR)

(72) Inventors: Cindy DOPIERALA, Paris (FR); Pierre-Yves GULERY, GRENOBLE (FR); Philippe CINQUIN, SAINT NAZAIRE LES EYMES (FR)

(21) Appl. No.: 17/631,778

(22) PCT Filed: Jul. 30, 2020
(86) PCT No.: PCT/EP2020/071535

§ 371 Date: Jan. 31, 2022

(30) Foreign Application Priority Data
Aug. 1, 2019 (FR) FR1908864

(51) Int. Cl. A61B 5/349; A61B 5/00; G16H 40/63

(52) U.S. Cl. CPC A61B 5/349; A61B 5/0031; A61B 5/686; A61B 5/6871; A61B 5/7275; G16H 40/63; A61B 2562/0219

(57) ABSTRACT

Disclosed is a device for determining a piece of information relating to a cardiac decompensation state of a user, the information being obtained by analysis of a cardiac parameter, characterized in that it includes a measuring device designed to determine a signal value by means of at least one accelerometer signal curve of the user, the signal value being intended to be compared with an additional signal value originating from a measurement by a cardiac monitor, the measuring device comprising, for this purpose, at least one accelerometer designed to determine said accelerometer signal curve, the measuring device being designed to be housed in an implant inside the user.

The field of the present invention is that of devices for measuring cardiac parameters of a human body.

In a known manner, heart failure can develop, in humans, into cardiac decompensation. Said cardiac decompensation leads to a change in the general state, with acute fatigue occurring even during rest, accompanied with the emergence of edema, which contributes to impairing the quality of gaseous exchanges of oxygen and carbon dioxide, which are essential to the human body.

A cardiac decompensation can thus lead to a cardiogenic edema in the region of the thorax, in particular in the pulmonary tissue. Respiratory problems and breathlessness are some of the symptoms, caused by compensation of the lungs, increasing their respiratory work and in particular causing chest pain. These symptoms worsen until the point of major respiratory distress if the accumulation of liquid is not detected earlier. Pulmonary edema is considered, medically, as a life-threatening emergency which must be treated with effect from the first symptoms, the treatments being all the more extensive the later the diagnosis, the tissues being less engorged at the initial stage.

Any patient at risk of heart disease must therefore be vigilant. The patients at risk are generally monitored by regular medical examinations, of the auscultation type, by the practitioner, an electrocardiogram, blood tests, and/or pulmonary radiography, in order to identify heart problems. In a preventative manner, the patient is forced to closely monitor their lifestyle and to treat their heart disease in order to avoid complications underlying cardiac decompensation.

However, medical monitoring remains restrictive for the patient, because they are dependent on the medical sector and the practitioner to perform an assessment of their pulmonary and cardiac state. Another disadvantage is the regularity of the monitoring, the patient having to frequently be subject to the medical sector in order to prevent worse complications. Furthermore, the monitoring cannot reasonably be performed over several days in the long term, for all patients, in particular those maintaining a good level of independence.

The aim of the present invention is therefore that of proposing a device that is capable of identifying the beginnings of a cardiac decompensation state, said device being simple to use and compatible with a repeated use for monitoring and early detection of cardiac problems in a patient at risk of cardiac decompensation.

The invention relates to a device for determining a piece of information relating to a cardiac decompensation state of a user, said information being obtained by analysis of a cardiac parameter, characterized in that it includes at least one measuring device designed to determine a signal value by means of at least one accelerometer signal curve of the user, said signal value being intended to be compared with an additional signal value originating from a measurement by a cardiac monitor, the measuring device comprising, for this purpose, at least one accelerometer designed to determine said accelerometer signal curve of the user, the measuring device being designed to be housed in an implant inside the user.

The device according to the invention is particularly advantageous in that it makes it possible to combine a bimodal analysis, taking into account accelerometric signals on the one hand and cardiac signals of the electrocardiogram type on the other hand, and an invasive measurement via implementation of at least one accelerometer in a suitable implant. Furthermore, it is notable that the information relating to a cardiac decompensation state is obtained in a simple manner by means of comparing two items of data, the simplification of the algorithm being able to facilitate the integration of corresponding calculation means in the implant, if applicable.

The electrocardiogram is a graphical representation of the electrical activity of the heart forming the basis of its mechanical activity, i.e. its contractions, while the mechanical activity in turn is monitored by means of the accelerometer.

By virtue of said implanted measuring device, the accelerometer signal curve and the cardiac parameter can be obtained in a reproducible and reliable manner. Indeed, the implantation participates in providing a fixed position of the accelerometer embedded in the implant, and at least in providing a stable position for measurement after measurement.

The measuring device comprises the accelerometer which generates the accelerometer signal curve. Said accelerometer signal curve is intended to be compared with other data, in this case to an electrocardiogram obtained by means of a cardiac monitor, in order to deduce therefrom the cardiac parameter. The accelerometer measures the acceleration according to at least one axis. The accelerometer is for example an accelerometer having from one to three mutually orthogonal axes.

The signal value, determined from the accelerometer signal curve, corresponds to a positive peak on the accelerometer signal curve, implying a maximum opening of the aortic valve of the user, referred to as the “aortic amplitude.” The appearance of a signal constitutes a time marker in the determination of the cardiac parameter.

According to one aspect of the invention, the determination device comprises a measuring means which is designed for measuring the additional signal value, said measuring means comprising at least the cardiac monitor, the determination device further comprising a calculation device designed for determining a value of the cardiac parameter depending on the time between the appearances of the signal and of the additional signal, the calculation device being designed to compare the cardiac parameter to a threshold value, exceeding which reveals a cardiac decompensation.

The cardiac monitor makes it possible to obtain an electrocardiogram trace which is the reflection of the electrical signal generated by cardiac activity of the user. In a manner complementary to what has been specified above regarding the fact that the measuring device is housed in the implant, the cardiac monitor can itself either also be embedded in the implant, or can be arranged externally with respect to the implant, and more particularly externally with respect to the user.

More particularly, the cardiac monitor can be external and portable, of the Holter monitor type, and continuously record the cardiac activity, in a relatively autonomous manner. The cardiac monitor may be an external scope that is not portable and requires the intervention of medical personnel. The cardiac monitor may be internal, for example embedded in the implant, thus operating autonomously once said implant is implanted.

The additional signal value is determined from the trace of the electrocardiogram. Said additional signal value corresponds to the wave R identified on the electrocardiogram, i.e. the second positive wave of the electrocardiogram, appearing after the wave P, which represents a ventricular depolarization of the user. The appearance of the additional signal constitutes a temporal marker in the determination of the cardiac parameter.

According to one feature of the invention, the cardiac parameter taken into consideration corresponds to a period of time, also referred to as the pre-ejection period or “PEP.” The cardiac parameter corresponding to the pre-ejection period is determined at least by virtue of the accelerometer signal curve and of the trace of the electrocardiogram, obtained simultaneously. The pre-ejection period corresponds to a time interval between the appearance of the wave R and the appearance of the maximum opening of the aortic valve. In other words, the cardiac parameter considered according to the invention and determined in a reproducible and reliable manner on account of the presence of the accelerometer in the implant is a time difference between the appearance of the additional signal determined on the electrocardiogram and the appearance of the signal determined on the accelerometer signal curve. Studying the values obtained by the cardiac monitor makes it possible to determine the start of the pre-ejection period, and studying the values obtained by the electrocardiogram makes it possible to determine the end of the pre-ejection period.

It will be understood that the device for determining a piece of information relating to a cardiac decompensation state of the user allows for a multimodal approach for obtaining the cardiac parameter. It makes it possible to compare a reliable and reproducible accelerometer signal curve with the trace of the electrocardiogram, in order to obtain the cardiac parameter. A comparison between the cardiac parameter and the threshold value makes it possible to assess the cardiac activity of the user, and to thus detect, early, a cardiac decompensation state. This comparison is carried out by the calculation device.

According to one aspect of the invention, the measuring device is designed to assume a position in the implant such that the accelerometer is capable of measuring at least an acceleration according to one axis from the dorsoventral axis, a lateral axis, and the rostro-caudal axis of the user. The accelerometer is an accelerometer having from one to three axes including at least one of said 3 axes.

According to one aspect of the invention, the measuring device is designed to be implanted in the user intragastrically. An implantation close to the user's heart, as is the case in intragastric implantation, makes it possible to obtain a specific accelerometer signal curve for the cardiac activity of the user. As has been mentioned, the electrocardiogram is a graphical representation of the electrical activity of the heart which forms the basis for the mechanical activity thereof. The cells of the heart muscle, under the impulse of a stimulation, depolarize and transmit the electrical impulse gradually through the heart. Measuring said electrical impulse may advantageously be carried out from the stomach, which is an organ located close to the heart. In order to achieve this, tow electrodes, a few centimeters apart from one another, are positioned so as to be in contact with the tissue of the gastric wall. They are connected to an integrated electronic module which conditions the signal measured by the electrodes. At a time t, each of the electrodes will measure a different potential. The measurement of said difference of potentials between the two electrodes, over time, results in an electrocardiogram.

For example, the measuring device is implanted intragastrically by means of endoscopy. For example, the measuring device is implanted so as to be fixed to the gastric wall or inserted into the gastric wall. For example, the measuring device is implanted in the top part of the stomach, in the region of or close to the stomach fundus.

According to one aspect of the invention, and as has been mentioned above, the cardiac monitor may be designed to be housed in the implant. “Housed in the implant” means that the monitor is embedded in the implant. The cardiac monitor can thus be largely housed within the implant, and can be connected to electrodes of the measuring means which are located on the surface of the implant or connected to the implant, it being necessary for the electrodes to be in contact with the tissues of the user.

The implant thus houses both the cardiac monitor included in the measuring means, and the measuring device comprising the accelerometer. In other words, the accelerometer signal curve and the electrocardiogram trace are obtained in the region of the implant, without an external connection. This contributes to obtaining an accelerometer signal curve and an electrocardiogram trace which are independent of a bias which is inherent in the variable positioning of the cardiac monitor and/or of the measuring device, since their positioning is fixed. The accelerometer signal curve and the electrocardiogram trace can be measured continuously and simultaneously, without the need for additional installation on the user. An arrangement of this kind makes it possible in particular to ensure that the measurement of the signals and additional signals is synchronized, controlling the triggering of these measurements in the same time base.

As mentioned above, the electrical and mechanical cardiac activities are linked. By means of temporal synchronization of the measurements made by the cardiac monitor contained in the measuring means, on the one hand, and by the measuring device comprising the accelerometer on the other hand, it is thus possible to analyze the electrical and mechanical activity of the heart in a concomitant manner, in order to reliably determine the cardiac parameter mentioned above.

For this purpose, the signals received are then pre-processed directly by the embedded processor of the device, or on the central server following transfer of the raw data. The pre-processing corresponds to filtering methods (Fourier transform, wavelet transform, empirical method, etc.), making it possible to improve the signal-to-noise ratio before analysis.

According to an alternative aspect of the invention, the cardiac monitor may be designed to be non-invasive. The cardiac monitor and the measuring means are physically separated from the measuring device. An arrangement of this kind makes it possible to propose a more compact implant, but it requires the provision of more complex synchronization means in order for the measurements of the signal, carried out in an invasive manner, and for those of the additional signal, carried out in a non-invasive manner, to share the same time base. The cardiac monitor must be installed specifically at the moment when it is wished to measure the electrical activity of the heart. The electrocardiogram is obtained by arranging measuring electrodes, connected by wires to the cardiac monitor, on the user's thorax. The measuring means is attached to the user, compared with the measuring device which, in the implant, is integrated into the user.

According to one aspect of the invention, the calculation device is designed to be housed in the implant. The calculation device is embedded together with the measuring device. This configuration facilitates the implementation of the determination device, and a repeated use thereof. The calculation device is for example designed to be arranged in the implant together with the accelerometer.

According to an alternative aspect of the invention, in which the calculation device is non-invasive and thus arranged at a distance from the implant, the measuring device comprises a communication member designed for transferring at least one signal to the calculation device. It will be understood that the communication member is associated both with the measuring device in the implant, i.e. at least with the accelerometer, and with the calculation device. The communication member comprises a transmitter for transferring the signal to a receiver included in the calculation device. For example, one or more transmitters of the communication member are embedded in the implant, and one or more receivers are designed to receive, in the region of the calculation device, the signal, with a view to externalized processing of said signal. The signal, received from the measuring device, comprises at least the accelerometer signals and/or a time marker corresponding to the appearance of the signal. When the measuring means, i.e. the means allowing for an electrocardiogram to be obtained, are housed in the implant, the signal transmitted by the communication member to the calculation device advantageously simultaneously comprises the accelerometer signals, the electrical signal generated by cardiac activity of the user, and/or a time marker corresponding to the appearance of the signal and/or a temporal marker corresponding to the appearance of the additional signal.

Various technologies can be used for connecting the transmitter and the receiver. By way of example, the following can be cited: wave-based, wireless communications technologies, such as a technology using Bluetooth or Wi-Fi.

According to one aspect of the invention, the implant comprises an energy storage device which is capable of supplying at least the measuring device. The energy storage device is advantageously inside the implant, and thus miniaturized. In one embodiment, it is a highly autonomous energy storage device, such as a long-life battery of the lithium-iodine battery type, which does not need to be connected to an external power source. In another embodiment, it is an energy storage device which is capable of being recharged wirelessly, from an external source. According to one aspect of the invention, the energy storage device is also capable of supplying the measuring means.

The invention also relates to a method for determining a piece of information relating to a cardiac decompensation state of a user, the determination method implementing the determination device as described above, during which a step of measuring signals makes it possible to obtain at least the signal value and the additional signal value, the signal value being obtained by the measuring device comprising at least the accelerometer, and the additional signal value being obtained by the measuring means comprising at least the cardiac monitor.

On the one hand, the step of measuring signals makes it possible to obtain the signal value. In order to achieve this, the accelerometer measures, during the step of measuring signals, the accelerometer signals so as to obtain the accelerometer signal curve. During the measuring step, the measuring device identifies, on the accelerometer signal curve, the maximum opening of the aortic valve of the user corresponding to the signal value.

On the other hand, the step of measuring signals makes it possible to obtain the additional signal value. In order to achieve this, the cardiac monitor measures, simultaneously with the step of measuring signals, and more particularly in a synchronous manner, i.e. proceeding from the same time base, the electrical activity generated by cardiac activity of the user, so as to obtain the electrocardiogram. During the measuring step, the measuring means identifies, on the electrocardiogram, the wave R corresponding to the additional signal value.

According to one aspect of the invention, the step of measuring signals is followed by a step of calculating a cardiac parameter which makes it possible to obtain the information on the cardiac decompensation state, said calculation step taking into account a time lag of the appearance of signals measured in the step of measuring signals. The time lag taken into account during said calculation step is read instantaneously on account of the synchronous measurement of the signal and of the additional signal, i.e. having a time base that is identical to the two signals. In other words, the accelerometer signal curve and the electrocardiogram are compared on the same time referential, so as to be able to obtain the cardiac parameter corresponding to the time lag between the appearance of the signal value and the appearance of the additional signal value. The wave R indicates the start of the pre-ejection period, and the maximum opening of the aortic valve of the user indicates the end of the pre-ejection period.

According to one aspect of the invention, the determination method comprises a step of calibration of the measuring device, the calibration step preceding the step of measuring signals. The calibration step makes it possible to obtain the threshold value, which is a reference value, exceeding which reveals a cardiac decompensation. The cardiac parameter is specific to each user, due to their cardiac activity, which is particular to the user, and due to the position of the measuring device, and particularly the position of the accelerometer, which may vary from one user to another and one implant to another. It should be noted that according to the invention, and the implantation of the accelerometer in the implant, said position of the measuring device is fixed and reproduced, over time, for a given user.

The calibration step makes it possible to personalize the determination method. In particular, it makes it possible to obtain a reference signal value specific to the user. It will be understood that “signal value” means an average of values which is sufficiently representative of the basal situation of the user.

According to one aspect of the invention, during the determination method, the step of calculating a cardiac parameter which makes it possible to obtain the information relating to the cardiac decompensation state is carried out not by an instantaneous approach, consisting in a calculation of a cardiac parameter for a signal value and a corresponding additional signal value, but by an averaged approach. More particularly, the measurement of the cardiac parameter corresponding to the pre-ejection period is performed on a coherent average representing the average of the signal acquired over 30 seconds, and thus also on an additional coherent average representing the average of the additional signal acquired synchronously over the same period.

According to one aspect of the invention, the result of the cardiac parameter calculation at a given moment, in particular by means of the averaged approach described above, without this being limiting, is recorded in an overall clinical picture. A daily clinical picture can also be generated, for the purpose of comparison with the preceding clinical pictures or a reference clinical picture obtained during the calibration step. More particularly, the value of the parameter on day D will be compared with that on day D−1, and the trace of the values over the course of the days will reflect a trend, either downwards or upwards, which may indicate a problem, or a stable line which indicate no major hemodynamic change.

According to an alternative aspect of the invention, the calculation device may compare, during the calculation step, the cardiac parameter with a threshold value, which can in particular be adjusted to the user during the calibration step. The calculation device manages the integration of parameters, including the cardiac parameter and the threshold value, in order to obtain the information relating to the cardiac decompensation state. The threshold value is deduced from the reference signal value. For example, the threshold value represents a percentage, variable depending on a calibration step prior to the implementation of the device, of the reference signal value.

During the calculation step, the cardiac parameter is both calculated and compared with the threshold value determined during the calibration step. The cardiac parameter/threshold value comparison makes it possible to finely determine the cardiac decompensation state of the user.

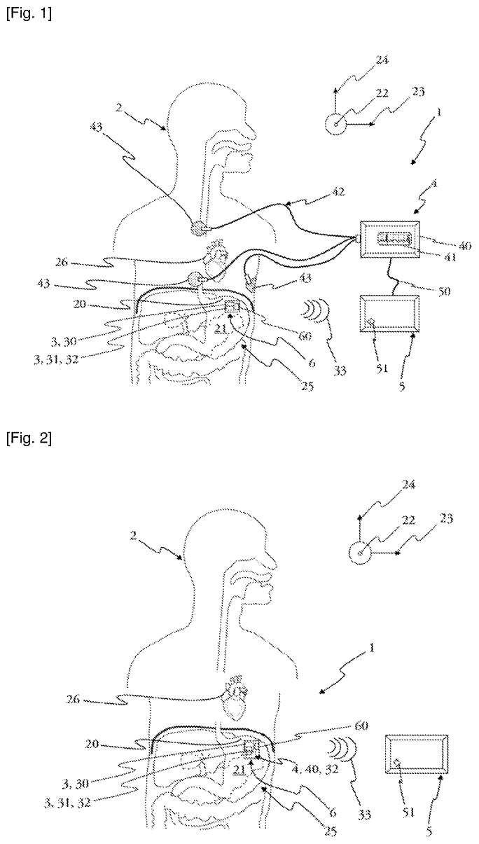

FIG. 1 is a general schematic view of a determination device according to the invention,

FIG. 2 is a general schematic view of a determination device according to the invention in another embodiment,

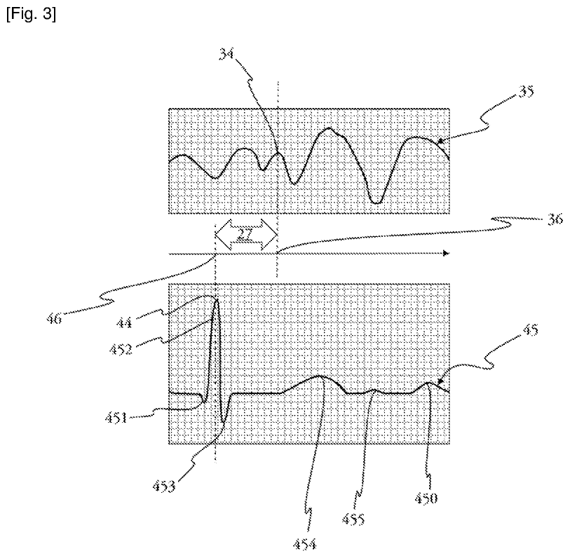

FIG. 3 illustrates a method for determining a piece of information relating to a cardiac decompensation state of a user, the determination method implementing the determination device according to the invention,

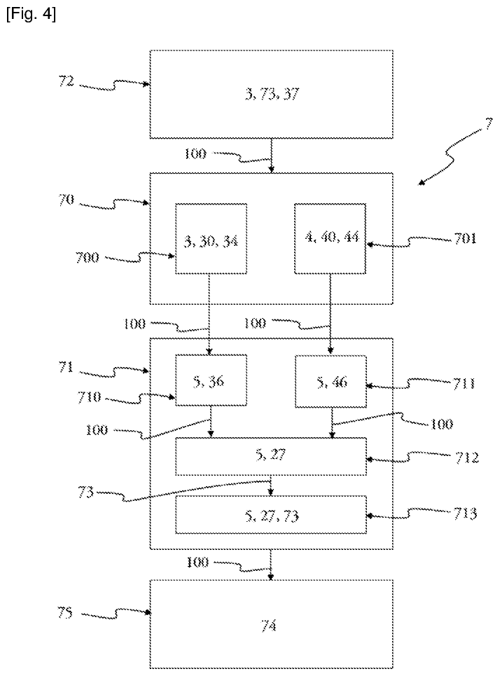

FIG. 4 is a flow diagram illustrating the determination method of FIG. 3.

It should firstly be noted that the figures disclose the invention in a detailed manner for implementing the invention, it of course being possible for said figures to serve to better define the invention, if applicable.

In the remainder of the description, the designations “internal/inside” and “external/outside” refer to the determination device according to the invention, and more particularly to an implant that forms part of said determination device. Any element integrated in an implant of the determination device is described as internal/inside or internalized, and any element located outside of the implant is described as external/outside or externalized.

Referring first to FIG. 1, a device 1 for determining a piece of information relating to a cardiac decompensation state of a user 2 is visible. The piece of information relating to a cardiac decompensation state is obtained by analyzing a cardiac parameter shown in FIG. 3.

The determination device 1 comprises at least one measuring device 3, a measuring means 4, and a calculation device 5. In the case in point, part of the determination device 1, comprising the measuring device 3, is internalized, and another part of the determination device 1, comprising the measuring means 4 and the calculation device 5, is externalized.

The measuring device 3 comprises at least one accelerometer 30 which is designed for determining an accelerometer signal curve of the user 2, shown in FIG. 3. The measuring device 3 is designed for determining a signal value, also shown in FIG. 3.

The measuring device 3 including the accelerometer 30 is housed in an implant 6 inside the user 2. The implant 6 corresponds to a hollow, biocompatible, and sealed compartment. The implant 6 is dimensioned so as to be implanted by means of an endoscopic device. The view of the implant 6 in FIG. 1 is schematic, and the implant 6 may be of any shape and dimension which is compatible with the implantation thereof, the function thereof, and the operation of the determination device 1. It will also be understood that the method of implantation of the implant is not considered here and can be implemented by any means.

The implant 6 is an intragastric implant which is positioned, in the embodiment of FIG. 1, in the region of the fundus 20 of the stomach 21 of the user 2, close to the heart 26 of the user 2. The implant 6 is for example fixed to the surface of a gastric mucous membrane or within the gastric mucous membrane, in the region of the fundus 20 of the stomach 21, or in the region of any tissue of the gastrointestinal tract 25.

The implant 6 comprises an energy storage device 60 which is capable of supplying at least the measuring device 3. The energy storage device 60 is miniaturized and isolated from the tissues of the user 2, such as in this case by being inside the implant 6. The energy storage device 60 is designed to have a service life of several years, so as to be able to supply the measuring device 3 as needed.

The measuring device 3 is designed to assume a position in the implant 6 such that the accelerometer 30 is capable of measuring at least an acceleration according to one axis from the dorsoventral axis 22, a lateral axis 23, and a rostro-caudal axis 24, as shown in FIG. 1.

The measuring means 4 is designed for measuring an additional signal value shown in FIG. 3, and comprises, for this purpose, at least one cardiac monitor 40. The cardiac monitor 40, which is externalized in this embodiment shown in FIG. 1, is equipped with a display screen 41 which makes it possible to display a trace of an electrocardiogram, as shown in FIG. 3.

The cardiac monitor 40 is connected, by wired sensors 42 of the measuring means 4, to a set of in this case three electrodes 43 which are each connected individually to the cardiac monitor 40 and fixed to the user 2. This representation of the measuring means 4 is not limiting, in particular with respect to the number of electrodes 43 used, it being possible for the measuring means 4 to assume any form as long as it makes it possible to measure the additional signal value.

The calculation device 5 is designed for determining a value of the cardiac parameter from the signal value and the additional signal value. The calculation device 5 can also be designed for comparing the cardiac parameter to a threshold value, exceeding which reveals a cardiac decompensation.

In the embodiment of FIG. 1, the calculation device 5 is external. It is on the one hand electrically connected, by cabling 50, to the cardiac monitor 40, so as to receive the additional signal value. It is on the other hand wirelessly connected to the measuring device 3. In order to achieve this, the measuring device 3 comprises a communication member 31 designed for transferring at least one signal to the calculation device 5. in this case the signal value. The measuring device 3 in particular comprises a transmitter 32 of waves 33, the transmitter 32 being inside the implant 6 and being connected to the measuring device 3. The calculation device 5 comprises a receiver 51 which is capable of receiving the waves 33 transmitted by the transmitter 32 of the communication member 31.

In the following, the various calculations carried out by the calculation device 5 in order to determine, according to the invention, a reliable piece of information relating to a possible cardiac decompensation state of the user, will be described, in particular with reference to FIGS. 3 and 4.

FIG. 2 shows another embodiment of the invention, the measuring means 4 being internal. In other words, the implant integrates both the accelerometer which forms the measuring device 3 for obtaining the signal value, and the cardiac monitor which forms the measuring means 4 for obtaining the additional signal value. In the embodiment shown, the determination device 1 is compacted in part, the measuring device 3 and the measuring means 4 being embedded in the implant 6, only the calculation device 5 being externalized in this case. The description of FIG. 1 applies, mutatis mutandis, to FIG. 2, and reference can be made thereto in order to understand and implement the invention.

In the embodiment of FIG. 2, the cardiac monitor 40 is designed to be housed in the implant 6. It is, just like the measuring device 3, supplied by the energy storage device 60. The communication member 31 is shared between the measuring device 3 and the measuring means 4, such that it transfers both the signals originating from the accelerometer 30, and the signals originating from the cardiac monitor 40. For example, the measuring device 3 and the measuring means 4 each comprise a transmitter 32 of the communication member 31 which transmits, via waves 33, the signal value and the additional signal value towards one or more receivers of the calculation device 5.

The fact that there is no calculation module integrated in the implant implies that all of the accelerometer signal and all of the signal of the electrical activity of the heart are transmitted to the calculation module remote from the implant. From these two transmitted signals, the calculation module performs a search of the opening point of the aortic valve, on the accelerometer signal, and of the maximum of the peak R, on the electrocardiogram, in order to find a temporal value of these two events and to subsequently perform the calculation of the time difference between said two signals defined on the same time base.

It will be understood that the interest of a doubly integrated device of this kind, i.e. having the accelerometer and the cardiac monitor housed in the implant, is that of facilitating the synchronization of the measurements and the sharing of the same time base for performing the two measurements simultaneously.

In this context, it is possible to provide for the calculation device to be integrated at least in part, or entirely, into the implant 6. In particular, it can be provided for part of the calculation to be performed in the implant, i.e. the measurement and the detection of the appearance of two time signals, and for it to be only the values of these time signals which are transmitted to an external database.

FIG. 3 shows different cardiac data, measured and determined by the determination device 1.

The signal value 34 corresponds to a time value that is determined by means of at least the accelerometer signal curve 35 of the user 2 obtained by the accelerometer 30. The accelerometer signal curve 35 comprises positive peaks and negative peaks, including a maximum opening of the aortic valve of the user 2. When said maximum opening of the aortic valve is identified, it is defined as corresponding to the signal value 34 on the accelerometer signal curve 35. The calculation device 5 derives therefrom a time marker 36, for example at a time t1 on a given time base.

The additional signal value 44 also corresponds to a time value that is determined by means of at least the electrocardiogram 45 of the user 2 obtained by the cardiac monitor 40. The electrocardiogram 45 comprises positive waves and negative waves, which can include a wave P 450, a wave Q 451, the wave R 452, a wave S 453, a wave T 454, a wave U 455. When the maximum of the wave R 452 is identified, it is defined as corresponding to the additional signal value 44 on the electrocardiogram 45. The calculation device 5 derives therefrom a temporal marker 46, for example at a time t0 on the time base common to that used for determining the first time marker 36.

The appearance of the signal 34 is intended to be compared chronologically to the appearance of the additional signal 44. In other words, the calculation device as described above is designed to calculate a time duration between the value of the time marker 36 and the value of the temporal marker 46. The value of the pre-ejection period, i.e. of the cardiac parameter 27, corresponds to that time period between the time marker 36 and the temporal marker 46. The calculation device 5 thus applies a formula according to which: [PEP=t1−t0]. It will be understood that, according to the invention, this determination is made easy by the synchronous measurement of the two signals, i.e. the user of the same time base for performing these two measurements.

FIG. 4 shows a flowchart which is representative of a method 7 for determining a piece of information relating to a cardiac decompensation state of a user 2, the determination method 7 implementing the determination device 1 as described above in FIG. 1. The determination method 7 comprises at least one step of measuring 70 signals, and a calculation step 71. Advantageously, the determination method 7 comprises at least one calibration step 72. Each step is represented by a rectangle in FIG. 4, the succession of steps being in accordance with a chronology indicated by the arrows 100.

The calibration step 72 makes it possible to calibrate at least the measuring device 3. It will be understood that the calibration step is theoretically performed once, before the device is operational for taking successive measurements, the aim of the calibration step being to fix a basal state, said state being kept for each of the measuring steps which will follow. Before embarking on the different measuring steps, a plurality of steps of calibration 72 of the measuring device 3 may be envisaged, for example during the implementation of the measuring device 3 post-implantation, or at a distance from the placement of the implant 6.

The calibration step 72 makes it possible to determine a threshold value 73 which is representative both of the baseline cardiac activity of the user 2, and of the positioning of the accelerometer 30. More particularly, the calibration step 72 consists in a multiplication of measurements of the cardiac parameter and the calculation of an average value of these measurements in order to derive therefrom the reference signal value 37. Proceeding from the reference signal value 37, a threshold value 73 corresponding to a percentage of the reference signal value 37 is determined, and said threshold value 73 is intended to be compared with the cardiac parameters 27 obtained during the measuring steps following the calibration step 72.

During the step of measuring signals 70, at least the signal value 34 and the additional signal value 44 are obtained. During a first measurement sub-step 700 included in the measuring step 70, the measuring device 3, and more particularly the accelerometer 30, is implemented in order to obtain the signal value 34. During a second measurement sub-step 701 included in the measuring step 70, the measuring means 4, and more particularly the cardiac monitor 40, is implemented in order to obtain the additional signal value 44. In the embodiment shown, the first measurement sub-step 700 and the second measurement sub-step 701 take place concomitantly, such that the values obtained can be compared on the same time referential so as to be able to calculate the cardiac parameter 27 during the calculation step 71. It will be understood that, without departing from the scope of the invention, said two sub-steps can be carried out in a staggered manner, in particular if the measurement of one of the signals may impair the measurement of the other signal.

The step of measuring 70 signals is followed by the step of calculating 71 the cardiac parameter 27, the calculation step 71 being implemented by the calculation device 5. The calculation step 71 may be performed periodically or regularly, or following each step of measuring 70 signals, and/or upon request by the user 2 or the medical staff.

The calculation step 71 makes it possible to identify the time marker 36 and the temporal marker 46 and to deduce therefrom the cardiac parameter 27. During a first calculation sub-step 710, the time marker 36 is identified. During a second calculation sub-step 711, the temporal marker 46 is identified.

During a third calculation sub-step 712 taking place after the first calculation sub-step 710 and the second calculation sub-step 711, the cardiac parameter 27 is deduced by the calculation device 5.

During a fourth calculation sub-step 713 of the calculation step 71, the calculation device 5 compares the cardiac parameter 27 with the threshold value 73 determined during the calibration step 72, or with a threshold value implemented in a theoretical manner in the calculation device.

At the end of the calculation step 71, a piece of information 74 on the cardiac decompensation state of the user 2 is obtained, during an information step 75. If the cardiac parameter 27 detected corresponds to a value above the threshold value, the user 2 is for example in an early state of cardiac decompensation and can be warned of this.

It will be understood, upon reading the above, that the present invention proposes a determination device which is designed to warn of an early state of cardiac decompensation. Said determination device, intended in particular to be implanted, at least in part, in the user, comprises at least one accelerometer which participates in determining a pre-ejection period, allowing for reliable detection of the signal value to be compared with an additional signal, the integration of said accelerometer in an implant allowing for said reliable measurement. The information obtained by a determination device of this kind is intended to be reliable and allows for frequent use, so as to ensure simple and recurrent monitoring of a user at risk of cardiac complications.

However, the invention is not limited to the means and configurations described and illustrated here, and it also extends to any equivalent means or configuration, and to any operational technical combination of such means. In particular, the form of the determination device may be modified without adversely affecting the invention, insofar as the determination device ultimately fulfils the same functions as those described in this document.

1. Device for determining a piece of information relating to a cardiac decompensation state of a user, said information being obtained by analysis of a cardiac parameter, characterized in that it includes at least one measuring device designed to determine a signal value by means of at least one accelerometer signal curve of the user, said signal value being intended to be compared with an additional signal value originating from a measurement by a cardiac monitor, the measuring device comprising, for this purpose, at least one accelerometer designed to determine said accelerometer signal curve of the user, the measuring device being designed to be housed in an implant inside the user.

2. Determination device according to claim 1, comprising a measuring means which is designed for measuring the additional signal value, said measuring means comprising at least the cardiac monitor, the determination device further comprising a calculation device designed for determining a value of the cardiac parameter depending on the time between the appearances of the signal and of the additional signal, the calculation device being designed to compare the cardiac parameter to a threshold value, exceeding which reveals a cardiac decompensation.

3. Determination device according to claim 1, wherein the measuring device is designed to assume a position in the implant such that the accelerometer is capable of measuring at least an acceleration according to one axis from the dorsoventral axis, a lateral axis, and a rostro-caudal axis.

4. Determination device according to claim 1, wherein the measuring device is designed to be housed in an intragastric implant inside the user.

5. Determination device according to claim 2, wherein the cardiac monitor is designed to be housed in the implant.

6. Determination device according to claim 2, wherein the calculation device is designed to be housed in the implant.

7. Determination device according to claim 2, wherein the measuring device comprises a communication member designed for transferring at least one signal to the calculation device.

8. Determination device according to claim 7, wherein the implant comprises an energy storage device which is capable of supplying at least the measuring device.

9. Method for determining a piece of information relating to a cardiac decompensation state of a user, the determination method implementing the determination device according to claim 1, during which a step of measuring signals makes it possible to obtain at least the signal value and the additional signal value, the signal value being obtained by the measuring device comprising at least the accelerometer, and the additional signal value being obtained by the measuring means comprising at least the cardiac monitor.

10. Determination method according to claim 9, implementing at least the calculation device, during which the step of measuring signals is followed by a step of calculating a cardiac parameter which makes it possible to obtain the information on the cardiac decompensation state, said calculation step taking into account a time lag of the appearance of the signals measured in the step of measuring signals.

11. Determination method according to claim 9, comprising a step of calibration of the measuring device, the calibration step preceding the step of measuring signals.

12. Determination method according to, claim 10 during which the calculation device compares, during the calculation step, the cardiac parameter with a threshold value determined during the calibration step.
